(19) United States
(12) Plant Patent Application Publication
(10) Pub. No.: US 2009/0113582 P1
(43) Pub. Date: Apr. 30, 2009

(54) Vriesea plant named 'Kiwi Sunset'

(50) ;

(76) Inventor: Andrew Maloy, Whenuapai (NZ)

Correspondence Address: HortisUSA, PO Box 3109, Camarillo, CA, 93011, US

(21) Appl. No.: 11/978,480

(22) Filed: Oct. 29, 2007

(51) Int. Cl. A01H 5/00

(52) U.S. Cl. PLT/263.1

(57) ABSTRACT

A new and distinct Vriesea cultivar named ‘Kiwi Sunset’ is disclosed, characterized by very stable, strongly reticulate variegated foliage of green/maroon. Upright plant form with a dark, pinkish heart.

### LATIN NAME OF THE GENUS AND SPECIES

Vriesea lindl.

### VARIETY DENOMINATION

‘Kiwi Sunset’

### BACKGROUND OF THE INVENTION

The new cultivar is a product of a planned breeding program. The new variety originated from a cross pollination of an unnamed, unknown seed parent and an unnamed, unknown pollen parent, both, Vriesea lindl. made by the inventor during 1996.

It was discovered by the inventor, Andrew Maloy, a citizen of New Zealand, in 1999 in a group of seedlings resulting from the 1996 crossing.

Asexual reproduction of the new cultivar ‘Kiwi Sunset’ was first performed in Auckland, New Zealand by tissue culture in 2002. ‘Kiwi Sunset’ has been reproduced by micro-propagation and has shown that the unique features of this cultivar are stable and reproduced true to type.

### SUMMARY OF THE INVENTION

The cultivar ‘Kiwi Sunset’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Kiwi Sunset’ These characteristics in combination distinguish ‘Kiwi Sunset’ as a new and distinct Vriesea cultivar:
1. Foliage resistant to stress.
2. Foliage is green with distinct bronze/purple transverse markings.
3. Paler coloring at leaf base.
4. Suitability as an indoor pot plant.
5. Pinkish hue to center of plant.
6. Broad leaves occurring in right, regular whorls.

Plants of the new cultivar ‘Kiwi Sunset’ cannot be compared to either seed parent or pollen parent as records of those characteristics were not kept. However, ‘Kiwi Sunset’ can be compared to the commercial variety Vriesea fosteriana ‘Red Chestnut.’ Plants of ‘Red Chestnut’ are similar to plants of ‘Kiwi Sunset’ in most horticultural characteristics, however, ‘Red Chestnut has a plain, uncolored heart and lighter green leaves than ‘Kiwi Sunset.’

‘Kiwi Sunset’ can also be compared to it seedling siblings, ‘Kiwi Cream’ and ‘Kiwi Dusk.’ Plant patent applications for these 2 varieties have been filed concurrently with that of ‘Kiwi Sunset.’ ‘Kiwi Sunset’ differs from ‘Kiwi Cream’ in having overall darker foliage and a pinkish center to the plant, whereas ‘Kiwi Cream’ has a cream colored center. ‘Kiwi Sunset’ differs from ‘Kiwi Dusk’ in having lighter green foliage and a center to the plant with more pink, rather than dark purple tones.

### BRIEF DESCRIPTION OF THE PHOTOGRAPH

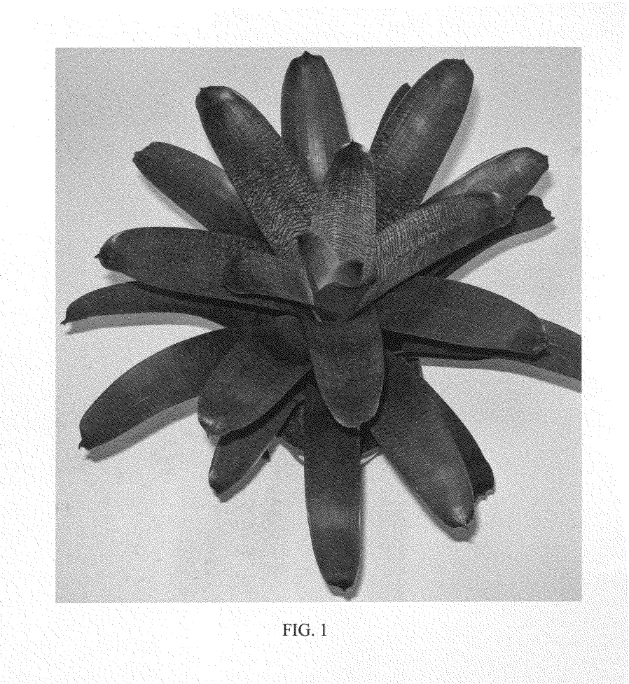

The accompanying photograph in FIG. 1 illustrates in full color a typical plant of ‘Kiwi Sunset’ grown in a greenhouse. This plant is approximately 4 years, 6 months old, shown in a 17 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘Kiwi Sunset’ plants grown in a climate controlled greenhouse in Saint Oedenrode, the Netherlands. Temperatures ranged from 18° C. to 22° C. at night to 18° C. to 22° C. during the day. No artificial light, photoperiodic treatments or chemical treatments were given to the plants. Measurements and numerical values represent averages of typical plant types.
Botanical classification: Vriesea lindl. cultivar ‘Kiwi Sunset.’

### PROPAGATION

Time to rooting: 2 months from a 2 year old tissue culture at approximately 20 to 22° C.
Root description: Fine, fibrous. Free Branching

### PLANT

Growth habit: Upright, forming compact rosettes around a single axis.
Plant shape: Inverted triangle.
Height: Approximately 29.6 cm.
Plant spread: Approximately 48.3 cm.
Normal pot size: 15 cm and larger.
Growth rate: Slow.
Branching characteristics: Upright, non-branching.
Number of leaves per stem: Approximately 30.
Age of plant described: Approximately 4 years, 6 months.

### FOLIAGE

Leaf:
   Arrangement.—Whorled.
   Average length.—Approximately 27.9 cm.
   Average width.—Approximately 6.2 cm.
   Shape of blade.—Oblong.
   Aspect.—Slightly recurved.
   Apex.—Sharply acute.
   Base.—Decurrent.
   Margin.—Entire.
   Texture of top surface.—Smooth, moderately glossy.
   Texture of bottom surface.—Smooth, moderately glossy.
   Color.—Young foliage upper side: Near R.H.S. Yellow-green 145C/D, flushed greyed-purple; 186A/B. Tip greyed-purple; 187A Upper half of the leaf blade has a reticulate variegation in the tone of, but, much darker than yellow-green 146A/147A. Towards the base of the leaf, this variegation is lighter; 144C/D. Young foliage under side: Near R.H.S. Greyed-purple 185C/D. Tip darker, near N186C. Base yellow-green; 150D Leaf blade has a reticulate variegation of greyed-purple N186C. This is lighter towards the base of the leaf, 183D. Mature foliage upper side: Upper half yellow-green 146B. Lower half greyed-green 193B/C. Tip greyed-purple 187A. Leaf blade has a reticulate variegation in the tone of yellow-green 146A/147A but much darker. Heavily flushed greyed-purple N186A towards the base of the leaf. Mature foliage under side: Near R.H.S. Greyed-purple 183A/B. Towards the base, coloration is lighter, 184A-B. Tip greyed-purple N186C. Whole surface color softened by a greyish layer. Leaf blade has a muddy(due to the greyish layer) reticulate variegation of greyed-purple N186B.
   Venation.—Type: Parallel. Venation coloration. Identical/Indistinguishable from foliage.
Petiole: Not present, sessile.
Flower: Flowering has not been observed, and is not commercially important in this cultivar.

### OTHER CHARACTERISTICS

Disease resistance: Neither resistance nor susceptibility to diseases or pests has been observed in this variety.
Drought tolerance and cold tolerance: Observed to tolerate temperatures to 35° C. without any negative effects. Temperature tolerance above this range has not been observed. Tolerance of cold temperatures have not been observed.
Fruit/seed production: Not observed.

What is claimed is:

1. A new and distinct cultivar of Vriesea plant named ‘Kiwi Sunset’ as herein illustrated and described.
